(19) United States
(12) Patent Application Publication
(10) Pub. No.: US 2017/0188882 A1
(43) Pub. Date: Jul. 6, 2017

(54) COILS AND COIL ASSEMBLIES THAT ARE RADIO-TRANSLUCENT

(71) Applicants: Boston Scientific Scimed Inc., Maple Grove, MN (US); Regents of the University of Minnesota, Minneapolis, MN (US)

(72) Inventors: Daniel J. Foster, Lino Lakes, MN (US); David R. Wulfman, Minneapolis, MN (US); Matthew Hein, Eden Prairie, MN (US); Kevin R. Poppe, New Brighton, MN (US)

(21) Appl. No.: 15/396,769

(22) Filed: Jan. 2, 2017

Related U.S. Application Data
(60) Provisional application No. 62/274,357, filed on Jan. 3, 2016.

(51) Int. Cl. A61B 5/06; H01F 7/06; H01F 41/04; H01F 27/02; H01F 27/32; H01F 41/12; A61B 6/00; H01F 27/28

(52) U.S. Cl. CPC A61B 5/062; A61B 6/503; H01F 7/06; H01F 27/2804; H01F 27/02; H01F 27/323; H01F 41/122; H01F 41/041

(57) ABSTRACT

A system that includes at least one coil that is radio-translucent and includes at least one conductor that includes lithium.

### CROSS-REFERENCE TO RELATED APPLICATION

This application claims priority to Provisional Application No. 62/274,357, filed Jan. 3, 2016, which is herein incorporated by reference in its entirety.

### TECHNICAL FIELD

The present disclosure relates to systems and methods for generating and sensing magnetic fields and, in particular, to coils and coil assemblies that are radio-translucent.

### BACKGROUND

Coils can be used to generate and sense magnetic fields in a variety of applications, such as transferring power from one device to another device, facilitating communications between devices, and tracking the positions of catheters in patients during various medical procedures.

In some systems, such as cardiac mapping and ablation systems, coils in a magnetic field generator generate magnetic fields that are sensed by coils in a catheter. The position of the catheter in the patient is determined from the sensed magnetic fields. Often, it is useful to simultaneously track the position of the catheter in the patient and obtain a radiograph, such as an x-ray and/or fluoroscopy, of the catheter in the patient.

Typically, the coils in the magnetic field generator are made out of copper, which is opaque to radiographs, such as x-rays and fluoroscopy. Since the coils are opaque to radiographs, they are located outside the field of view of the radiograph to prevent visually obstructing the image of the catheter and the patient. Since magnetic fields degrade significantly with distance, the coils must generate strong magnetic fields to be effective. This can result in larger coils, fewer coils, non-uniform magnetic fields, weaker magnetic field gradients, difficulty in shaping the magnetic fields, more complex sensor systems, and more metal-in-field effects.

### SUMMARY

Example 1 is a system including at least one coil that is radio-translucent and includes at least one conductor that includes lithium.

Example 2 is the system of Example 1, wherein the at least one conductor is a lithium conductor that is wrapped around a core of the at least one coil.

Example 3 is the system of any of Examples 1 and 2, wherein the at least one conductor is insulated by at least one of a thin film insulator and a silicone insulator.

Example 4 is the system of any of Examples 1-3, wherein the at least one coil includes a coating to prevent moisture ingress.

Example 5 is the system of Example 1, wherein the at least one coil is a planar coil.

Example 6 is the system of any of Examples 1-5, including at least one of a coil frame and a substrate that supports the at least one coil.

Example 7 is the system of Example 6, wherein the at least one of the coil frame and the substrate is radio-translucent and at least a portion of the at least one of the coil frame and the substrate and the at least one coil have a uniform radio-translucency in at least one direction.

Example 8 is the system of any of Examples 6 and 7, including at least one vapor barrier that is radio-translucent and one of vacuum sealed and filled with an inert gas and sealed, wherein one of: the at least one of the coil frame and the substrate and the at least one coil are situated inside the at least one vapor barrier and the at least one vapor barrier is hermetically sealed to prevent ingress of moisture; and each coil of the at least one coil is situated in a separate vapor barrier of the at least one vapor barrier and the separate vapor barrier is hermetically sealed to prevent ingress of moisture.

Example 9 is the system of Example 8, including a housing that is radio-translucent, wherein the at least one vapor barrier is situated in the housing.

Example 10 is the system of any of Examples 1-9, wherein different coils of the at least one coil are oriented in at least two different directions.

Example 11 is a method of manufacturing a system, the method including at least one of: insulating at least one conductor that includes lithium and winding the at least one conductor around a core to provide a coil that is radio-translucent; and disposing a conductive trace that includes lithium on a substrate to provide a planar coil that is radio-translucent.

Example 12 is the method of Example 11, including at least one of: disposing the coil into a coil frame that is radio-translucent such that the coil and at least a portion of the coil frame have a uniform radio-translucency in at least one direction; and disposing the conductive trace on the substrate such that the planar coil and at least a portion of the substrate have a uniform radio-translucency in at least one direction.

Example 13 is the method of Example 12, including inserting a plug that is radio-translucent into a core of the coil to provide the uniform radio-translucency in the at least one direction.

Example 14 is the method of any of Examples 12 and 13, including at least one of: inserting at least one of the coil frame and the substrate into a vapor barrier that is radio-translucent, hermetically sealing the vapor barrier to prevent moisture ingress, and inserting the vapor barrier into a housing that is radio translucent; and inserting the coil into a separate vapor barrier that is radio-translucent, hermetically sealing the separate vapor barrier to prevent moisture ingress, and inserting the separate vapor barrier into the housing that is radio-translucent.

Example 15 is the method of any of Examples 11-14, wherein insulating the at least one conductor comprises at least one of insulating the at least one conductor with a thin film insulator and insulating the at least one conductor with a silicone insulator.

Example 16 is a system including a plurality of coils, wherein the plurality of coils are radio-translucent to x-rays and include conductors that include lithium.

Example 17 is the system of Example 16, including a coil frame that supports the plurality of coils, wherein the coil frame and the plurality of coils provide a uniform radio-translucent image to x-rays in at least one direction.

Example 18 is the system of Example 16, wherein each of the plurality of coils includes a conductor of the conductors that is electrically insulated by at least one of a thin film insulator and a silicone insulator.

Example 19 is the system of Example 16, wherein at least one of the plurality of coils is a planar coil that includes a conductive trace that includes lithium.

Example 20 is the system of Example 16, including a housing that is radio-translucent, wherein the plurality of coils are situated inside the housing.

Example 21 is the system of Example 20, wherein the housing is at least one of a box, a patch, a paddle, and a plate that is configured to be at least one of mounted to a platform and situated on a patient.

Example 22 is the system of Example 16, including at least one vapor barrier that is radio-translucent, wherein one of: the plurality of coils are situated inside the at least one vapor barrier and the at least one vapor barrier is hermetically sealed to prevent ingress of moisture; and each of the plurality of coils is individually situated inside a separate vapor barrier of the at least one vapor barrier and the separate vapor barrier is hermetically sealed to prevent ingress of moisture.

Example 23 is the system of Example 22, wherein the at least one vapor barrier is at least one of vacuum sealed, filled with an inert gas and sealed, and an aluminized bag.

Example 24 is the system of Example 22, including a housing that is radio-translucent, wherein the at least one vapor barrier is situated inside the housing.

Example 25 is a system including a coil assembly, including a coil frame and a plurality of coils that are radio-translucent and supported by the coil frame. The system includes a vapor barrier that is radio-translucent, wherein at least one of the plurality of coils is situated in the vapor barrier, and a housing that is radio-translucent, wherein the vapor barrier is situated in the housing.

Example 26 is the system of Example 25, wherein the coil frame and the plurality of coils provide a uniform radio-translucent image in at least one direction.

Example 27 is the system of Example 25, wherein the plurality of coils are configured for at least one of generating a magnetic field and sensing a magnetic field and the plurality of coils are configured to be at least one of individually activated and activated as part of a group of the plurality of coils.

Example 28 is the system of Example 25, wherein all of the plurality of coils are oriented in the same direction.

Example 29 is the system of Example 25, wherein different coils of the plurality of coils are oriented in at least two different directions.

Example 30 is a method of manufacturing a system, the method including: providing a plurality of coils by at least one of: insulating a conductor that includes lithium and winding the conductor around a core to provide a coil that is radio-translucent; and disposing a conductive trace that includes lithium on a substrate to provide a planar coil that is radio-translucent.

Example 31 is the method of Example 30, including disposing the plurality of coils in a coil frame to provide a coil assembly that provides a uniform radio-translucency in at least one direction.

Example 32 is the method of Example 31, including inserting a plug that is radio-translucent in a core of one or more of the plurality of coils to provide the uniform radio-translucency.

Example 33 is the method of Example 30, wherein insulating a conductor comprises at least one of insulating the conductor with a thin film insulator and insulating the conductor with a silicone insulator.

Example 34 is the method of Example 30, including at least one of: inserting the plurality of coils into a vapor barrier that is radio-translucent and hermetically sealing the vapor barrier to prevent moisture ingress; and inserting each of the plurality of coils into a separate vapor barrier that is radio-translucent and hermetically sealing each of the separate vapor barriers to prevent moisture ingress.

Example 35 is the method of Example 34, including at least one of: inserting the vapor barrier with the plurality of coils into a housing that is radio-translucent; and inserting at least one of the separate vapor barriers into the housing that is radio-translucent.

While multiple embodiments are disclosed, still other embodiments of the disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an electro-anatomical mapping system that includes a magnetic field generator, according to embodiments of the disclosure.

FIG. 2 is a diagram illustrating a coil assembly, according to embodiments of the disclosure.

FIG. 3 is a diagram illustrating a cross-section of the coil assembly taken along the line A-A in FIG. 2, according to embodiments of the disclosure.

FIG. 4 is a diagram illustrating a coil that is radio-translucent, according to embodiments of the disclosure.

FIG. 5A is a diagram illustrating a coil that is radio-translucent and that includes an insulated ribbon conductor and a core, according to embodiments of the disclosure.

FIG. 5B is a cross-section of the insulated ribbon conductor taken along the line B-B in FIG. 5A, according to embodiments of the disclosure.

FIG. 6 is a diagram illustrating a coil that is radio-translucent and includes multiple layers of material laminated together to make a spiral electromagnetic coil 150, according to embodiments of the disclosure.

FIG. 7A is a graph illustrating the transmission of x-rays through different materials versus the thickness of the materials at 10 kilo-electronVolts (KeV) of energy.

FIG. 7B is a graph illustrating the transmission of x-rays through the different materials versus the thickness of the materials at 50 KeV of energy.

FIG. 7C is a graph illustrating the transmission of x-rays through the different materials versus the thickness of the materials at 100 KeV of energy.

FIG. 8A is a graph illustrating the attenuation of x-rays through the different materials versus the thickness of the materials at 10 KeV of energy.

FIG. 8B is a graph illustrating the attenuation of x-rays through the different materials versus the thickness of the materials at 50 KeV of energy.

FIG. 8C is a graph illustrating the attenuation of x-rays through the different materials versus the thickness of the materials at 100 KeV of energy.

FIG. 9 is a diagram illustrating a coil assembly that includes coils situated in three different directions in a coil frame that is radio-translucent, according to embodiments of the disclosure.

FIG. 10 is a diagram illustrating a cross-section of the coil assembly taken along the line C-C in FIG. 9, according to embodiments of the disclosure.

FIG. 11A is a diagram illustrating the coil situated with its core and longitudinal axis parallel to (in) the x-direction, according to embodiments of the disclosure.

FIG. 11B is a diagram illustrating the coil situated with its core and longitudinal axis at an angle a1 relative to the x-direction in the x-y plane, according to embodiments of the disclosure.

FIG. 11C is a diagram illustrating the coil situated with its core and longitudinal axis at an angle a2 relative to the x-direction in the x-y plane, according to embodiments of the disclosure.

FIG. 12 is a diagram illustrating a coil assembly, according to embodiments of the disclosure.

FIG. 13A is a diagram illustrating one example of a cross-section of the coil assembly taken along the line D-D in FIG. 12, according to embodiments of the disclosure.

FIG. 13B is a diagram illustrating another example of a cross-section of coil assembly 300 taken along the line D-D in FIG. 12, according to embodiments of the disclosure.

FIG. 14 is a diagram illustrating a coil assembly and a bag that is radio-translucent, according to embodiments of the disclosure.

FIG. 15 is a diagram illustrating a housing that is radio-translucent with the bag and the coil assembly of FIG. 14 situated inside the housing, according to embodiments of the disclosure.

FIG. 16 is a diagram illustrating a coil that is radio-translucent and a bag that is radio-translucent, according to embodiments of the disclosure.

FIG. 17 is a diagram illustrating a housing that is radio-translucent, which encloses a coil assembly that is radio translucent, according to embodiments of the disclosure.

FIG. 18 is a diagram illustrating a paddle that is radio-translucent, according to embodiments of the disclosure.

FIG. 19 is a diagram illustrating a patch that is radio-translucent, according to embodiments of the disclosure.

FIG. 20 is a diagram illustrating a method of manufacturing a system that includes coils that are radio-translucent, according to embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

The present disclosure describes embodiments of systems and methods that include coils for generating and/or sensing magnetic fields. In the various embodiments, the coils are effectively radio-translucent or radiolucent, so that the coils can be situated in the field of view of a radiograph, such as an x-ray and/or fluoroscopy, without significantly affecting the contrast of the radiographic image of the point of interest. The coils and, optionally, a coil frame or substrate that supports the coils, are more radio-transparent than the point of interest.

In a magnetic field generator, the coils can be situated closer to the point of interest, such that the generated field strength can be less, the coils can be smaller and/or lighter weight, and more coils can be situated near the point of interest. This allows for a more uniform magnetic field active area and greater control over magnetic field shaping and dynamic coil activation. Also, the closer coils can provide a stronger magnetic field gradient that leads to greater spatial resolution. In addition, more coils and local fields lead to less metal-in-field effects and less complex sensor designs. In some embodiments, the coils and, optionally, a coil frame or substrate that supports the coils, provide a uniform image in at least one direction, which is more radio-transparent than the point of interest.

Throughout this disclosure, coils are shown and described as being substantially round or circular coils, however, the coils described herein can have different shapes and are not limited to being round or circular. For example, in some embodiments, coils can be rectangular, oblong, oval, square, or otherwise have a different shape. Also, in at least some embodiments, the coils are one example of an antenna.

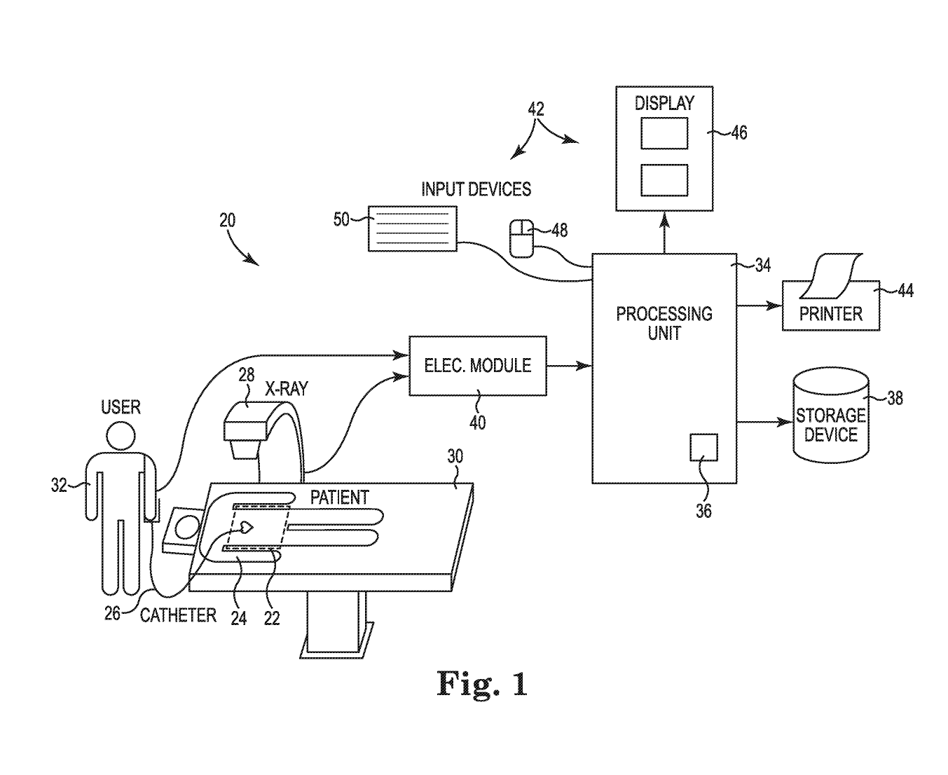

FIG. 1 is a diagram illustrating an electro-anatomical mapping system 20 that includes a magnetic field generator 22, according to embodiments described in the disclosure. The electro-anatomical mapping system 20 is capable of mapping cardiac rhythms of a patient 24 and, in some embodiments, generating and displaying a three-dimensional map of the cardiac geometry as well as various cardiac electrical activity parameters. The system 20 includes a catheter 26 having one or more electrodes for measuring electrical activity in the heart of the patient 24 and one or more coils for sensing magnetic fields. In some embodiments, the catheter 26 includes three different coils disposed at three different angles for sensing the magnetic fields.

The magnetic field generator 22 generates one or more magnetic fields that are sensed by the coil(s) in the catheter 26. Signals from the coil(s) associated with the sensed magnetic fields are used to track the location of the catheter 26 in the patient 24. The system 20 also includes radiographic equipment 28 for taking radiographs, such as x-rays and/or fluoroscopy, of the patient 24 lying on the table 30. The radiographs can be taken as part of the mapping procedure, where the magnetic field generator 22 does not obstruct or significantly affect the contrast of the radiographic image of the point of interest. With information obtained via the catheter 26 and the radiographic images, a user 32, such as a physician or a technician, and/or the system 20 itself maps the cardiac rhythms of the patient 24.

In one example of a signal acquisition stage, the catheter 26 is displaced to multiple locations within the heart chamber of interest into which the catheter 26 is inserted. At each of these locations, the electrodes on the catheter 26 measure the electrical activity of the heart. Also, the magnetic field generator 22 generates one or more magnetic fields that are sensed by the coil(s) on the catheter 26, and the location coordinates of the catheter 26 in the chamber of interest are determined from the sensed magnetic fields. In addition, radiographs are taken as the electrodes measure the electrical activity of the heart at the different locations. The electrical measurements, coordinates of the catheter 26, and radiographs are analyzed together to map the heart chamber. In some embodiments, the catheter 26 is configured for contact mapping. In some embodiments, the catheter 26 is configured for near-contact mapping. In some embodiments, the catheter 26 is configured for non-contact mapping.

In some embodiments, the electrodes are mounted on the catheter 26 following a three dimensional olive shape, where the electrodes are mounted on a device capable of deploying the electrodes into the desired shape while inside the heart and capable of retracting the electrodes when the catheter is removed from the heart. To allow deployment into the three dimensional shape, the electrodes may be mounted on a balloon or shape memory material, such as Nitinol. Also, in some embodiments, the catheter 26 may include any number of electrodes arranged according to any desired shape.

In some embodiments, the system 20 includes other catheters and/or electrodes for measuring electrical activity of the heart. These electrodes can be externally mounted on or near the patient 24 for measuring the electrical activity of the heart. For example, the system 20 can include electrocardiogram (ECG or EKG) leads that are used to measure the electrical activity of the heart. The system 20 can use the signals obtained from the ECG leads for system functions, such as determining which new or existing mapping configurations more closely match a cardiac rhythm and switching to a different mapping configuration for adding data to an associated cardiac map.

At each of the locations to which the catheter 26 is moved, the catheter's one or more electrodes acquire signals resulting from the electrical activity of the heart. This provides, to the user 32, physiological data pertaining to the heart's electrical activity based on information acquired at multiple locations, which may facilitate, for example, providing a relatively accurate reconstruction of the physiological behavior of the endocardium surface. The acquisition of signals at multiple catheter locations in the heart chamber enables the catheter 26 to effectively act as a “mega-catheter” whose effective number of electrodes and electrode span is proportional to the product of the number of locations in which signal acquisition is performed and the number of electrodes on the catheter 26. In some embodiments of near-contact and non-contact mapping, to enhance the quality of the physiological information at the endocardium surface, the catheter 26 is moved to more than three locations, such as more than 5, 10, or even 50 locations, within the heart chamber. Further, the spatial range over which the catheter is moved may be larger than one third (⅓) of the diameter of the heart cavity, such as larger than 35%, 40%, 50% or even 60% of the diameter of the heart cavity.

In some embodiments, the physiological information is computed based on signals measured over several heart beats, either at a single catheter location within the heart chamber or over several locations. In circumstances where physiological information is based on multiple measurements over several heart beats, the measurements can be synchronized with one another so that the measurements are performed, and/or analyzed, with respect to approximately the same phase of the heart cycle. Also, the signal measurements over multiple beats can be synchronized based on features detected from physiological data, such as, for example, a surface electrocardiogram (ECG) or an intracardiac electrogram (EGM).

The system 20 includes a processing unit 34, which may be, or include, a processor that executes code stored in internal memory 36 and/or in a storage device 38 to perform operations, according to embodiments of the disclosure. The internal memory 36 and/or the storage device 38 also, or alternatively, may store data acquired by the one or more electrodes of the mapping catheter 26 and the radiographic equipment 28. In some embodiments, the internal memory 36 and/or the storage device 38 may store data acquired via other catheters and or external electrodes, such as via ECG leads. In some embodiments, the processing unit 34 is an electronic processor, which may be, at least in part, a software processor.

The processing unit 34 is communicatively coupled to the catheter 26 and the radiographic equipment 28 to receive the signals from the one or more electrodes and the image data from the radiographic equipment 28. The processing unit 34 executes, from memory such as the internal memory 36 and/or the storage device 38, computer code for mapping the cardiac rhythms of the patient 24. In some embodiments, an automated set-up routine processes the data to determine new mapping configurations and may provide at least some set-up information and/or set-up results for the new mapping configurations to the user 32. In some embodiments, the processing unit 34 executes code that processes the data with beat detection and beat acceptance criteria for one or more existing mapping configurations.

In some embodiments, the processing unit 34 executes a reconstruction procedure to determine the physiological information at the endocardium surface. To expedite embodiments of computational operations performed by the system 20, the processing unit 34 may compute, prior to the insertion of the catheter 26 into the heart chamber and/or before signal acquisition by the catheter's electrodes has commenced, transformation functions that can be used, during a mapping procedure, to facilitate the reconstruction process. Once the catheter 26 is inserted and displaced to a particular location in the heart chamber, the mapping procedure may be performed expeditiously by computing those transformation components that were not computed ahead of the signal acquisition stage, and combining those components with the appropriate pre-processed transformation components to obtain the overall transformation function(s). The overall transformation function may be applied to the acquired raw data to perform an inverse reconstruction operation.

The processing unit 34 performs a catheter registration procedure. The location of the catheter 26 inserted into the heart chamber is determined using the sensing and tracking system of the magnetic field generator 22 and the coil(s) of the catheter 26. This tracking system also provides three dimensional spatial coordinates of the catheter 26 and/or its multiple electrodes with respect to the catheter's coordinate system as established by the sensing and tracking system. Also, to perform the mapping procedure and/or reconstruct physiological information on the endocardium surface, it is desirable to align the coordinate system of the catheter 26 with the endocardium surface's coordinate system. The processing unit 34 or another processing module of the system 20 is configured to determine a coordinate system transformation function that transforms the three dimensional spatial coordinates of the catheter's locations into coordinates expressed in terms of the endocardium surface's coordinate system, or vice-versa. The radiographs obtained by the radiographic equipment 28 can be used to derive the coordinate system transformation function and in other aspects of the catheter registration procedure. In some embodiments, the processing unit 34 performs post-processing operations on the reconstructed physiological information to extract and display useful features of the information to the operator of the system 20 and/or other persons, such as a physician.

The signals acquired by the one or more electrodes of the catheter 26 and the image data acquired by the radiographic equipment 28 can be passed to the processing unit 34 through an electronic module 40. The module 40 receives the signals and performs signal enhancement operations on them before they are forwarded to the processing unit 34. Signal conditioning hardware may be used to amplify, filter, and sample intracardiac potentials measured by one or more electrodes. In some embodiments, for example, the intracardiac signals have maximum amplitudes of 60 mV and mean amplitudes of a few millivolts. In some embodiments, the signals are bandpass filtered in a frequency range, such as 0.5-500 Hz, and sampled with analog to digital converters, such as converters with 15-bit resolution at 1 kHz.

To avoid interference with electrical equipment in the room, the signals may be filtered to remove one or more frequencies corresponding to the equipment. Other types of signal processing operations may be implemented, such as, for example, spectral equalization, automatic gain control, and/or the like. The resultant processed signals are forwarded by the module 34 to the processing unit 28 for further processing. In some embodiments, the image data acquired by the radiographic equipment 28 is passed directly from the radiographic equipment 28 to the processing unit 34.

The system 20 includes a user interface 42 and, optionally, peripheral devices, such as a printer 44, which are communicatively coupled to the processing unit 34. The user interface 42 includes one or more display devices 46 and input devices, such as a mouse 48 and a keyboard 50. The display devices 46 receive signals from the processing unit 34 and display cardiac maps and information related to the cardiac maps, including physiological and electrical activity data about the patient 24. In some embodiments, the user interface 42 includes a graphical user interface that includes a touch screen.

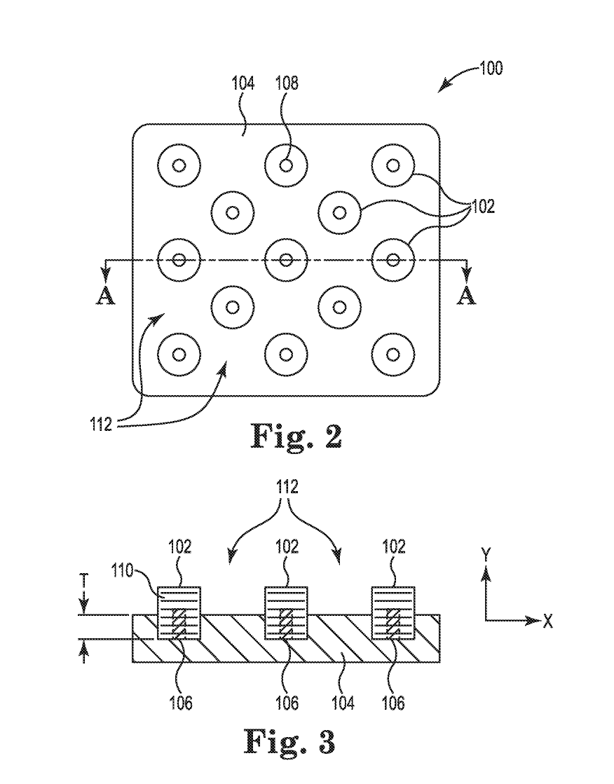

FIG. 2 is a diagram illustrating a coil assembly 100, according to embodiments of the disclosure. In the various embodiments, the coil assembly 100 is effectively radio-translucent and includes coils 102 that are radio-translucent and disposed in and supported by a coil frame 104, which in the various embodiments is also effectively radio-translucent, at least in the regions that are positioned within the X-ray or fluoroscopic field of interest in a medical procedure. Also, since the coil assembly 100 is radio-translucent, it can be situated in the field of view of a radiograph, such as an x-ray and/or fluoroscopy, without significantly affecting the contrast of the radiographic image of the point of interest, where the coil assembly 100 is more radio-transparent than the point of interest. In some embodiments, the magnetic field generator 22 (shown in FIG. 1) includes the coil assembly 100.

In various embodiments, the coil frame 104 supports the plurality of coils 102 and is made from one or more radio-translucent materials. In some embodiments, the coil frame 104 includes polypropylene. In some embodiments, the coil frame 104 includes high-density polyethylene (HDPE).

The coils 102 can be used for generating a magnetic field and/or sensing a magnetic field. Each of the coils 102 is electrically coupled to an electronic controller, such as processing unit 34 (shown in FIG. 1). In some embodiments, each of the coils 102 can be individually activated to generate localized magnetic fields and/or sense magnetic fields. In some embodiments, the coils 102 can be activated in groups of two or more and, in some embodiments, all of the coils 102 can be activated at the same time.

FIG. 3 is a diagram illustrating a cross-section of the coil assembly 100 taken along the line A-A in FIG. 2, according to embodiments of the disclosure. The coils 102 are situated in and supported by the coil frame 104. In this example, all of the coils 102 are oriented in the same direction, with the longitudinal axis of each of the coils 102 aligned with, i.e., parallel with, the y-direction, indicated in FIG. 3. In other embodiments, different coils of the coils 102 are oriented in two or more directions, such as the x-direction and the y-direction and/or other directions. In some embodiments, different coils of the coils 102 are oriented in three different directions.

In some embodiments, the coil assembly 100 provides a uniform radio-translucency, i.e., a uniform radio-translucent image, in at least one direction. In this example, the coil assembly 100 provides a uniform radio-translucent image in the y-direction. To provide this uniform radio-translucent image in the y-direction, the thickness T of the radio-translucent coil frame 104, from the bottom of the coils 102 to the top of the coil frame 104, is configured to provide the same radio-translucency in the y-direction as that provided by the coils 102 in the y-direction. In addition, radio-translucent material, such as polypropylene and/or HDPE is inserted as a plug 106 into the core 108 (shown in FIG. 2) of each of the coils 102 to provide the same radio-translucency in the y-direction as that provided by the coils 102 in the y-direction.

The coils 102 include conductors 110, such as ribbons or wires, wound around the core 108. The conductors 110 are made out of one or more radio-translucent materials. In some embodiments, the conductors 110 include lithium. In some embodiments, the conductors 110 include a lithium alloy. In some embodiments, the conductors 110 include aluminum. In some embodiments, the conductors 110 include beryllium. In some embodiments, the conductors 110 include carbon nano-tubes. In some embodiments, the coils 102 include a lithium coil that includes lithium conductors, such as ribbons or wires, which are insulated by at least one of a thin film insulator and a silicone insulator.

During operation, the coils 102 conduct current to generate one or more magnetic fields and/or to sense one or more magnetic fields, such that the coils 102 generate heat due to current-induced heating. To reduce heating of the coils 102 and/or the frame 104, one or more active and/or passive cooling techniques may be employed.

In some embodiments, a cooling fluid 112 is disposed to flow around the coils 102 and/or the frame 104 to reduce heating. The cooling fluid 112 is circulated around the coils 102 and/or the frame 104 by at least one of a passive mechanism such as a convection current mechanism or an active mechanism such as a cooling fan or a cooling pump. In some embodiments, the frame 104 is made out of a porous material or includes channels that the cooling fluid 112 flows through to cool the coils 102 and/or the frame 104. In some embodiments, the cooling fluid 112 is air. In some embodiments, the cooling fluid 112 is a liquid, such as a non-reactive mineral oil, a liquid that boils at atmospheric pressure or has a low boiling point such as Freon and R134A, or another liquid such as polyisobutylene, hexane, and heptane.

In some embodiments, the coils 102 and/or the frame 104 can be cooled by inserting wax around the coils 102 to wick away the heat. Also, in some embodiments, the coils 102 and/or the frame 104 can be cooled by putting fins on the frame 104, where the air and/or fluid flows around the fins to dissipate the heat.

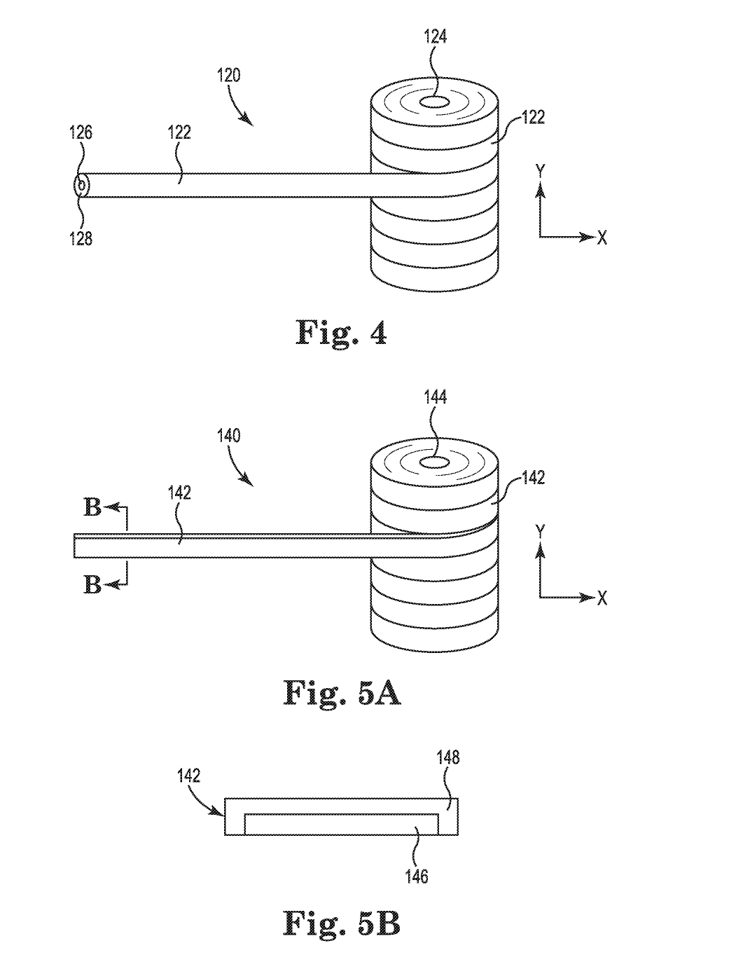

FIG. 4 is a diagram illustrating a coil 120 that is radio-translucent, according to embodiments of the disclosure. The coil 120 includes an insulated conductor 122 and a core 124. The insulated conductor 122 is wound around the core 124. In some embodiments, the core 124 is centrally located in the coil 120, such that the insulated conductor 122 is evenly wound, i.e., distributed, around the core 124. In some embodiments, the coil 120 is disposed in the coil frame 104 (shown in FIGS. 2 and 3). In some embodiments, at least one, and up to all, of the coils 102 (shown in FIGS. 2 and 3) is similar to the coil 120.

The insulated conductor 122 is radio-translucent and includes a conductor 126 and an insulator 128. The conductor 126 is made from one or more conductive radio-translucent materials and can be in the shape of a circular wire. The insulator 128 coats the conductor 126. In some embodiments, the conductor 126 is centrally located in the insulated conductor 122, such that the insulator 128 is evenly distributed around the conductor 126. In some embodiments, the conductor 126 includes lithium. In some embodiments, the conductor 126 includes a lithium alloy. In some embodiments, the conductor 126 includes aluminum. In some embodiments, the conductor 126 includes beryllium. In some embodiments, the conductor 126 includes carbon nano-tubes.

The insulator 128 is disposed or applied onto the conductor 126 and insulates the conductor 126 from itself as the insulated conductor 122 is wound around the core 124. The insulator 128 is radio-translucent. In some embodiments, the insulator 128 is made from or at least includes silicone.

The core 124 is made from one or more radio-translucent materials. In some embodiments, the core 124 is made from the same material(s) as the coil frame 104. In some embodiments, the core 124 includes polypropylene. In some embodiments, the core 124 includes HDPE. In addition, in some embodiments, one or more radio-translucent materials, such as polypropylene and/or HDPE, is inserted as a plug (not shown) into the core 124 to provide the same radio-translucency in the y-direction as that provided by the insulated conductor 122 in the y-direction.

FIG. 5A is a diagram illustrating a coil 140 that is radio-translucent and that includes an insulated ribbon conductor 142 and a core 144, according to embodiments of the disclosure. The insulated ribbon conductor 142 is wound around the core 144. In some embodiments, the core 144 is centrally located in the coil 140, such that the insulated ribbon conductor 142 is evenly wound, i.e., distributed, around the core 144. In some embodiments, the coil 140 is disposed in the coil frame 104 (shown in FIGS. 2 and 3). In some embodiments, at least one, and up to all, of the coils 102 (shown in FIGS. 2 and 3) is similar to the coil 140.

FIG. 5B is a cross-section of the insulated ribbon conductor 142 taken along the line B-B in FIG. 5A. In reference to FIGS. 5A and 5B, the insulated ribbon conductor 142 is radio-translucent and includes a conductor 146 and an insulator 148. The conductor 146 is made from one or more conductive radio-translucent materials and is in the shape of a ribbon. The insulator 148 coats the conductor 146 on three sides. In some embodiments, the conductor 146 includes lithium. In some embodiments, the conductor 146 includes a lithium alloy. In some embodiments, the conductor 146 includes aluminum. In some embodiments, the conductor 146 includes beryllium. In some embodiments, the conductor 146 includes carbon nano-tubes.

The insulator 148 is disposed on three sides of the conductor 146 and insulates the conductor 146 from itself as the insulated ribbon conductor 142 is wound around the core 144. The insulator 148 is radio-translucent. In some embodiments, the insulator 148 is made from a thin film insulator. In some embodiments, the insulator 148 is made from or at least includes silicone.

The core 144 is made from one or more radio-translucent materials. In some embodiments, the core 144 is made from the same material(s) as the coil frame 104. In some embodiments, the core 144 includes polypropylene. In some embodiments, the core 144 includes HDPE. In addition, in some embodiments, one or more radio-translucent materials, such as polypropylene and/or HDPE, is inserted as a plug (not shown) into the core 144 to provide the same radio-translucency in the y-direction as that provided by the insulated ribbon conductor 142 in the y-direction.

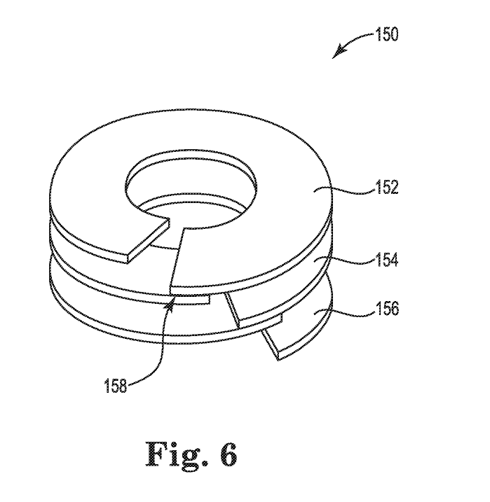

FIG. 6 is a diagram illustrating a coil 150 that is radio-translucent and includes multiple layers of material laminated together to make a spiral electromagnetic coil 150, according to embodiments of the disclosure. The coil 150 includes multiple split donut conductive portions 152, 154, and 156, where, in some embodiments, each of the split donut conductive portions 152, 154, and 156 is disposed on a separate radio-translucent substrate. The substrates are laminated together and the split donut conductive portions 152, 154, and 156 are connected together, such as at 158, to make a spiral coil 150. In some embodiments, the substrates are insulating substrates. In some embodiments, the spiral coil 150 is similar to a Bitters electromagnetic coil.

The split donut conductive portions 152, 154, and 156 are made from one or more conductive radio-translucent materials. In some embodiments, one or more of the split donut conductive portions 152, 154, and 156 includes lithium. In some embodiments, one or more of the split donut conductive portions 152, 154, and 156 includes a lithium alloy. In some embodiments, one or more of the split donut conductive portions 152, 154, and 156 includes aluminum. In some embodiments, one or more of the split donut conductive portions 152, 154, and 156 includes beryllium. In some embodiments, one or more of the split donut conductive portions 152, 154, and 156 includes carbon nano-tubes.

In some embodiments, a radio-translucent insulator coats one or more of the split donut conductive portions 152, 154, and 156. In some embodiments, the insulator is made from a thin film insulator. In some embodiments, the insulator is made from or at least includes silicone.

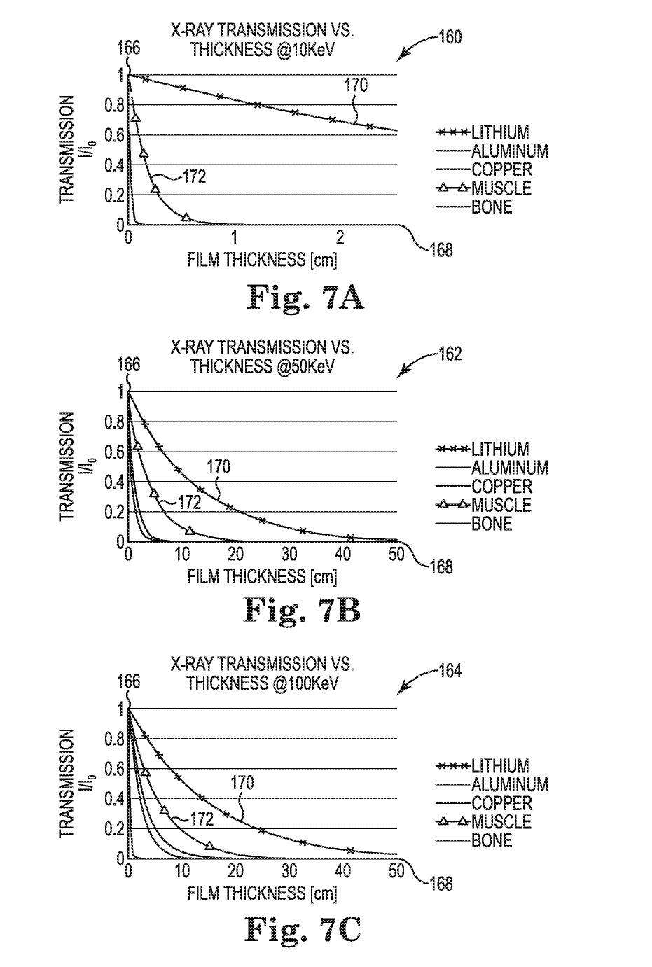

FIGS. 7A-7C are graphs 160, 162, and 164 illustrating the transmission of x-rays through different materials versus the thickness of the materials, at three different energy levels of the x-rays. In each graph, the transmission of x-rays is measured as the ratio of the x-rays passing through the material versus the x-rays originally passing into the material, I/Io, on a scale from 0 to 1 on the y-axis at 166. The thickness is measured in centimeters (cm) on the x-axis 168. The materials include lithium 170 and muscle 172, along with aluminum, copper, and bone.

FIG. 7A is a graph 160 illustrating the transmission of x-rays through the different materials versus the thickness of the materials at 10 kilo-electronVolts (KeV) of energy. As shown, lithium at 170 has a much greater radio-translucency, i.e., permits a greater percentage of the x-rays to pass through, than any of the other materials.

FIG. 7B is a graph 162 illustrating the transmission of x-rays through the different materials versus the thickness of the materials at 50 KeV of energy. As shown, lithium at 170 has a much greater radio-translucency, i.e., permits a greater percentage of the x-rays to pass through, at thicknesses between 0 and 50 cm, than any of the other materials.

FIG. 7C is a graph 164 illustrating the transmission of x-rays through the different materials versus the thickness of the materials at 100 KeV of energy. As shown, lithium at 170 has a much greater radio-translucency, i.e., permits a greater percentage of the x-rays to pass through, at thicknesses between 0 and 50 cm, than any of the other materials.

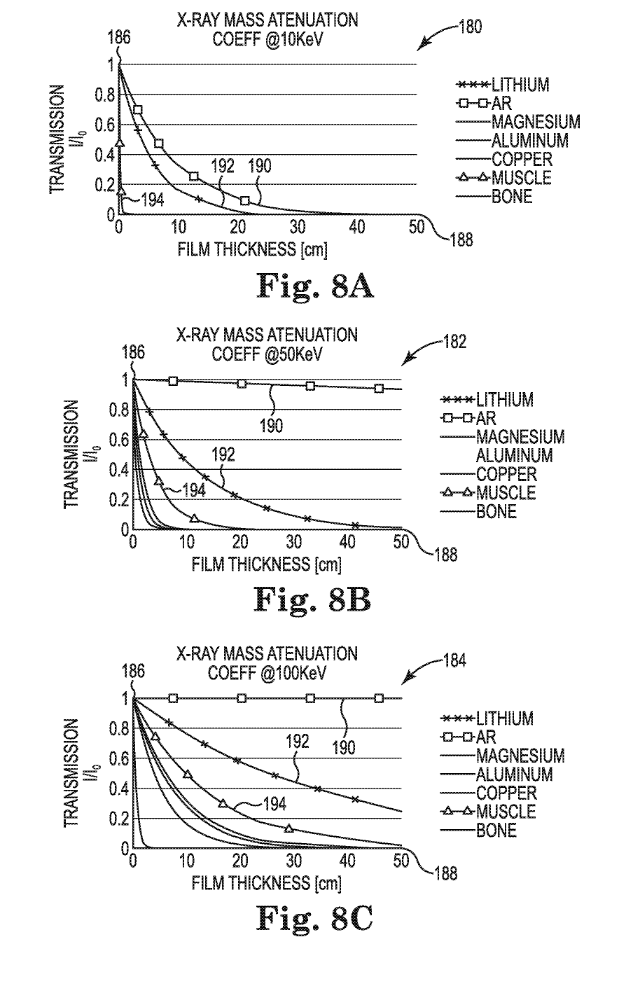

FIGS. 8A-8C are graphs 180, 182, and 184 illustrating the attenuation of x-rays through different materials versus the thickness of the materials, at three different energy levels of the x-rays. In each graph, the attenuation of x-rays is measured as the ratio of the x-rays passing through the material versus the x-rays originally going into the material, I/Io, on a scale from 0 to 1 on the y-axis at 186. The thickness is measured in millimeters (mm) on the x-axis 188. The materials include argon 190, lithium 192, and muscle 194, along with magnesium, aluminum, copper, and bone.

FIG. 8A is a graph 180 illustrating the attenuation of x-rays through the different materials versus the thickness of the materials at 10 KeV of energy. As shown, argon at 190 attenuates less than lithium 192, which attenuates less than any of the other materials from at least 0-20 mm of thickness.

FIG. 8B is a graph 182 illustrating the attenuation of x-rays through the different materials versus the thickness of the materials at 50 KeV of energy. As shown, argon at 190 attenuates less than lithium 192, which attenuates less than any of the other materials from at least 0-20 mm of thickness.

FIG. 8C is a graph 184 illustrating the attenuation of x-rays through the different materials versus the thickness of the materials at 100 KeV of energy. As shown, argon at 190 attenuates less than lithium 192, which attenuates less than any of the other materials from at least 0-20 mm of thickness.

As shown in the graphs of FIGS. 7A-7C and the graphs of FIGS. 8A-8C, lithium has a radio-translucency that is greater than muscle and bone. This makes lithium good for use in coils that may be or are placed in the viewing area of a radiograph, such as an x-ray and/or fluoroscopy, where the coils do not prevent the viewing of a patient or instruments, such as a catheter, used on the patient.

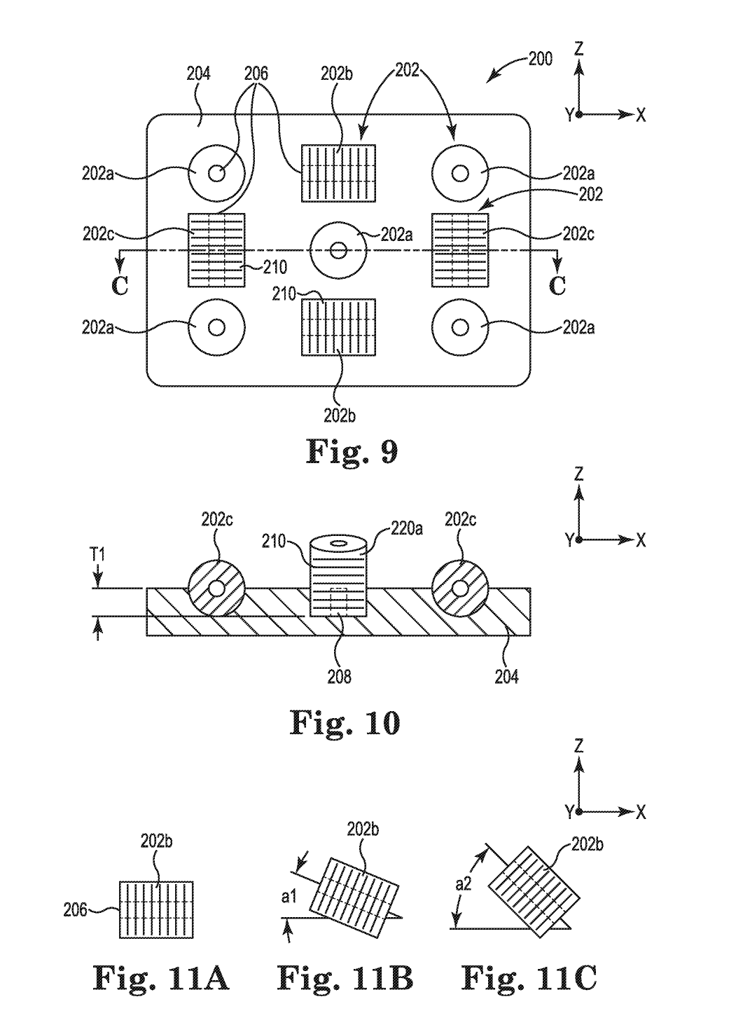

FIG. 9 is a diagram illustrating a coil assembly 200 that includes coils 202 situated in three different directions in a coil frame 204 that is radio-translucent, according to embodiments of the disclosure. The coil assembly 200 is radio-translucent and can be used for generating and/or sensing magnetic fields. Also, since the coil assembly 200 is radio-translucent, it can be situated in the field of view of a radiograph, such as an x-ray and/or fluoroscopy, without significantly affecting the contrast of the radiographic image of the point of interest, where the radio-translucent coil assembly 200 minimizes attenuation in addition to that resulting from the materials of the point of interest, such as a patient and the patient's muscle and/or bone. In some embodiments, the magnetic field generator 22 (shown in FIG. 1) includes the coil assembly 200.

The coil frame 204 supports the plurality of coils 202 and is made from one or more radio-translucent materials. In some embodiments, the coil frame 204 includes polypropylene. In some embodiments, the coil frame 204 includes HDPE.

Each of the coils 202 includes a core 206 that is parallel to its longitudinal axis. The coils 202 are situated in three different directions in the coil frame 204. The coils 202a are situated with their cores 206 and longitudinal axes in (parallel to) the y-direction, which is into and out of the page in FIG. 9, and at 90 degrees to the x-direction and the z-direction. The coils 202b are situated with their cores 206 and longitudinal axes in the x-direction, and at 90 degrees to the y-direction and the z-direction. The coils 202c are situated with their cores 206 and longitudinal axes in the z-direction, and at 90 degrees to the x-direction and the y-direction. In other embodiments, one or more of the coils 202 can be angled from the x-direction, the y-direction, and the z-direction at non-zero angles, not parallel to any of the x-direction, the y-direction, or the z-direction. In other embodiments, the coils 202 can be situated in two directions or in more than three directions.

The coils 202 can be used for generating a magnetic field and/or sensing a magnetic field. Each of the coils 202 is electrically coupled to an electronic controller, such as processing unit 34 (shown in FIG. 1). In some embodiments, each of the coils 202 can be individually activated to generate localized magnetic fields and/or sense magnetic fields. In some embodiments, the coils 202 can be activated in groups of two or more, such as a group including coils 202a, a group including coils 202b, and another group including coils 202c. In some embodiments, all of the coils 202 can be activated at the same time.

FIG. 10 is a diagram illustrating a cross-section of the coil assembly 200 taken along the line C-C, according to embodiments of the disclosure. The coils 202 are situated in and supported by the coil frame 204. As shown in FIG. 10, the single radio-translucent coil 202a is situated with its core 206 and longitudinal axis in the y-direction and the two coils 202c are situated with their cores 206 and longitudinal axes in the z-direction, into and out of the page in FIG. 10.

In some embodiments, the coil assembly 200 has a uniform translucency, i.e., provides a uniform radio-translucent image, in at least one direction. To provide a uniform radio-translucent image in the y-direction, the thickness T1 of the radio-translucent coil frame 204, from the bottom of the coils 202 to the top of the coil frame 204, is configured to provide the same radio-translucency in the y-direction as that provided by the coils 202 in the y-direction. In addition, radio-translucent material, such as polypropylene and/or HDPE is inserted as a plug 208 into the cores 206 of at least the coils 202a to provide the same radio-translucency in the y-direction as that provided by the coils 202a in the y-direction.

The coils 202 include conductors 210, such as ribbons or wires, wound around the core 206. The conductors 210 are made out of one or more radio-translucent materials. In some embodiments, the conductors 210 include lithium. In some embodiments, the conductors 210 include a lithium alloy. In some embodiments, the conductors 210 include aluminum. In some embodiments, the conductors 210 include beryllium. In some embodiments, the conductors 210 include carbon nano-tubes. In some embodiments, the coils 202 include a lithium coil that includes lithium conductors, such as ribbons or wires, which are insulated by at least one of a thin film insulator and a silicone insulator. In some embodiments, the coils 202 are similar to one or more of the coils 120 and 140 of FIGS. 4-6.

FIGS. 11A-11C are diagrams illustrating one of the coils 202b situated at three different angles in relation to the x-direction in the x-y plane, according to embodiments of the disclosure. In FIGS. 11A-11C, the radio-translucent coil 202b is situated at 90-degrees in relation to the z-direction. In other examples, the coil 202b can be situated at angles other than 90 degrees in relation to the z-direction.

FIG. 11A is a diagram illustrating the coil 202b situated with its core 206 and longitudinal axis parallel to (in) the x-direction, according to embodiments of the disclosure. FIG. 11B is a diagram illustrating the coil 202b situated with its core 206 and longitudinal axis at an angle a1 relative to the x-direction in the x-y plane, according to embodiments of the disclosure. FIG. 11C is a diagram illustrating the coil 202b situated with its core 206 and longitudinal axis at an angle a2 relative to the x-direction in the x-y plane, according to embodiments of the disclosure. Having one or more coils 202 at different angles in the coil assembly 200 enhances the ability of the coil assembly 200 to generate and/or sense differently orientated magnetic fields.

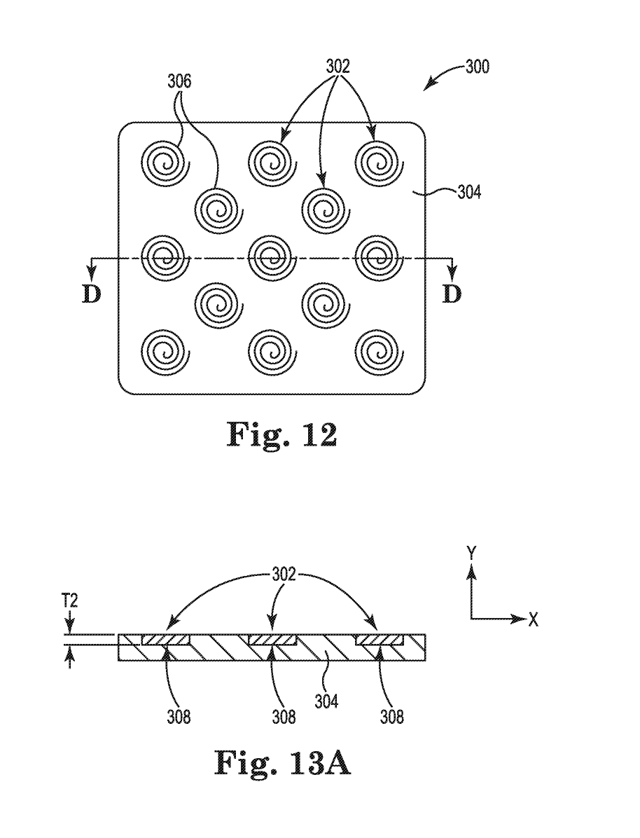

FIG. 12 is a diagram illustrating a coil assembly 300, according to embodiments of the disclosure. The coil assembly 300 is radio-translucent and includes coils 302 that are radio-translucent and in or on a coil frame 304 that is radio-translucent. The coil assembly 300 can be used for generating and/or sensing magnetic fields. Also, since the coil assembly 300 is radio-translucent, it can be situated in the field of view of a radiograph, such as an x-ray and/or fluoroscopy, without significantly affecting the contrast of the radiographic image of the point of interest, where the radio-translucent coil assembly 300 minimizes attenuation in addition to that resulting from the materials of the point of interest, such as a patient and a patient's muscle and/or bone. In some embodiments, the magnetic field generator 22 (shown in FIG. 1) includes the coil assembly 300.

The coil frame 304 supports the plurality of coils 302 and is made from one or more radio-translucent materials. In some embodiments, the coil frame 304 includes polypropylene. In some embodiments, the coil frame 304 includes HDPE.

The coils 302 can be used for generating a magnetic field and/or sensing a magnetic field. Each of the coils 302 is electrically coupled to an electronic controller, such as processing unit 34 (shown in FIG. 1). In some embodiments, each of the coils 302 can be individually activated to generate localized magnetic fields and/or sense magnetic fields. In some embodiments, the coils 302 can be activated in groups of two or more and, in some embodiments, all of the coils 302 can be activated at the same time.

The coils 302 are disposed in or on the coil frame 304. The coils 302 include traces or conductors 306 made out of one or more radio-translucent materials. In some embodiments, the conductors 306 include lithium. In some embodiments, the conductors 306 include a lithium alloy. In some embodiments, the conductors 306 include aluminum. In some embodiments, the conductors 306 include beryllium. In some embodiments, the conductors 306 are carbon nano-tubes. In some embodiments, the coils 302 include a lithium coil that includes lithium conductors 306.

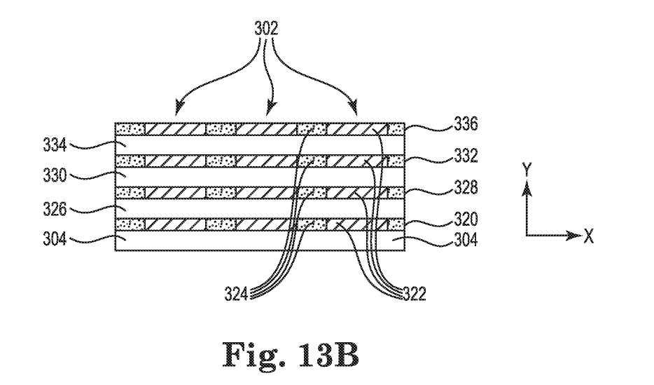

FIG. 13A is a diagram illustrating one example of a cross-section of the coil assembly 300 taken along the line D-D in FIG. 12, according to embodiments of the disclosure. The coils 302 are disposed on indented surfaces 308 in the coil frame 304 and supported by the coil frame 304. In this example, all of the surfaces 308 are parallel and, subsequently, all of the coils 302 are oriented in the same direction. In other embodiments, the surfaces 308 can be oriented at different angles to one another and different coils of the coils 302 can be oriented at different angles in the coil frame 304, such as in two or more directions. In some embodiments, different coils of the coils 302 are oriented in three different directions.

In some embodiments, the coil assembly 300 has a uniform radio-translucency, i.e., provides a uniform radio-translucent image, in at least one direction. In this example, the coil assembly 300 provides a uniform radio-translucent image in the y-direction. To provide this uniform radio-translucent image in the y-direction, the thickness T2 of the radio-translucent coil frame 304, from the bottom of the coils 302 to the top of the coil frame 304, is configured to provide the same radio-translucency in the y-direction as that provided by the coils 302 in the y-direction. In addition, radio-translucent material, such as polypropylene and/or HDPE is disposed between the traces or conductors 306 of a radio-translucent coil 302 to provide the same radio-translucency in the y-direction as that provided by the coils 302 in the y-direction.

FIG. 13B is a diagram illustrating another example of a cross-section of coil assembly 300 taken along the line D-D in FIG. 12, according to embodiments of the disclosure. In this example, the coil assembly 300 includes a laminated structure that includes multiple layers of material laminated together and on the coil frame 304 to form three dimensional coils 302. In some embodiments, the laminated structure can be embedded in the coil frame 304.

In some embodiments, a first coil layer 320 including radio-translucent coil conductors 322 separated by an insulator and/or photomask material 324 is laminated on the coil frame 304. A first separator layer 326, such as a circuit board layer or an insulating layer, is laminated on the first coil layer 320. A second coil layer 328 including radio-translucent coil conductors 322 separated by an insulator and/or a photomask material 324 is laminated on the first separator layer 326, and a second separator layer 330, such as a circuit board layer or an insulating layer, is laminated on the second coil layer 328. A third coil layer 332 including radio-translucent coil conductors 322 separated by an insulator and/or a photomask material 324 is laminated on the second separator layer 330, and a third separator layer 334, such as a circuit board layer or an insulating layer, is laminated on the third coil layer 332. A fourth coil layer 336 including radio-translucent coil conductors 322 separated by an insulator and/or a photomask material 324 is laminated on the third separator layer 334. The vertically aligned coil conductors 322 are electrically coupled together, in series, to form the different three dimensional coils 302.

In this example, all of the coils 302 are oriented in the same direction. In other embodiments, different coils of the coils 302 can be oriented at different angles in or on the coil frame 304, such as in two or more directions. In some embodiments, different coils of the coils 302 are oriented in three different directions.

In some embodiments, the coil assembly 300 has a uniform radio-translucency, i.e., provides a uniform radio-translucent image, in at least one direction. In this example, the coil assembly 300 provides a uniform radio-translucent image in the y-direction. To provide this uniform radio-translucent image in the y-direction, the insulator and/or a photomask material 324 is configured to provide the same radio-translucency in the y-direction as that provided by the coil conductors 322 in the y-direction. In addition, radio-translucent material, such as polypropylene and/or HDPE is disposed between the traces or conductors 322 of the radio-translucent coils 302 to provide the same radio-translucency in the y-direction as that provided by the coils 302 in the y-direction.

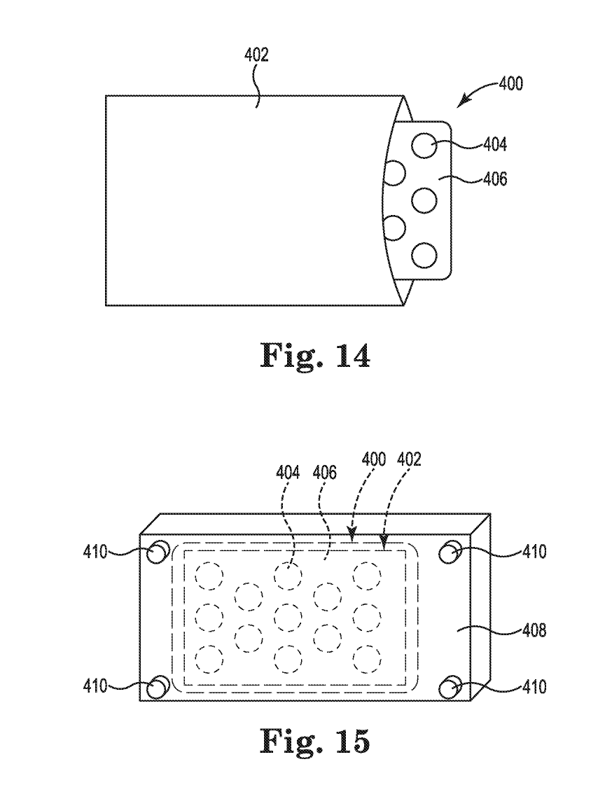

FIG. 14 is a diagram illustrating a coil assembly 400 and a bag 402 that is radio-translucent, according to embodiments of the disclosure. The coil assembly 400 is radio-translucent and inserted into the bag 402, such that the bag 402 with the coil assembly 400 inside of it is radio-translucent. Thus, the bag 402 with the coil assembly 400 inside can be situated in the field of view of a radiograph, such as an x-ray and/or fluoroscopy, without significantly affecting the contrast of the radiographic image of the point of interest, where the radio translucent bag 402 with the coil assembly 400 inside minimizes attenuation in addition to that resulting from the materials of the point of interest, such as a patient and the patient's muscle and/or bone. The bag 402 is one example of a vapor barrier that surrounds or encapsulates a coil and/or the coil assembly 400 to prevent ingress of moisture that would otherwise damage the coil or the coil assembly 400. In some embodiments, the bag 402 includes plastic. In some embodiments, the bag 402 is an aluminized bag, i.e., a bag at least coated with a layer of aluminum. In some embodiments, the magnetic field generator 22 (shown in FIG. 1) includes the coil assembly 400 inside the bag 402.

The coil assembly 400 includes coils 404 that are radio-translucent in a coil frame 406 that is radio-translucent. The coil assembly 400 can be used for generating and/or sensing magnetic fields. Also, since the coil assembly 400 is radio-translucent, it can be situated in the field of view of a radiograph, such as an x-ray and/or fluoroscopy, without significantly affecting the contrast of the radiographic image of the point of interest, where the radio-translucent coil assembly 400 minimizes attenuation in addition to that resulting from the materials of the point of interest, such as a patient and the patient's muscle and/or bone. In some embodiments, the coil assembly 400 is similar to one or more of: the coil assembly 100 of FIG. 1; the coil assembly 200 of FIG. 9; and the coil assembly 300 of FIG. 12.

The coil assembly 400 is inserted into the bag 402 and the bag 402 is hermetically sealed. Hermetically sealing the bag 402 prevents ingress of moisture into the bag 402, moisture that would otherwise damage the coil assembly 400. In some embodiments, the bag 402 is vacuum sealed. In some embodiments, the bag 402 is filled with an inert gas, such as argon or nitrogen, and then hermetically sealed. In some embodiments, the bag 402 is filled with a fluid, such as cooling fluid 112, to cool the coil assembly 400.

FIG. 15 is a diagram illustrating a housing 408 that is radio-translucent with the bag 402 and the coil assembly 400 of FIG. 14 situated inside the housing 408, according to embodiments of the disclosure. The housing 408 with the bag 402 and coil assembly 400 inside of it is radio-translucent. Thus, the housing 408 with the bag 402 and the coil assembly 400 can be situated in the field of view of a radiograph, such as an x-ray and/or fluoroscopy, without significantly affecting the contrast of the radiographic image of the point of interest, where the radio-translucent housing 408 with the radio-translucent bag 402 and the radio-translucent coil assembly 400 minimizes attenuation in addition to that resulting from the materials of the point of interest, such as a patient and the patient's muscle and/or bone. In some embodiments, the housing 408 includes and/or is made of carbon fiber. In some embodiments, the magnetic field generator 22 (shown in FIG. 1) includes the radio-translucent housing 408 with the radio-translucent bag 402 and the radio-translucent coil assembly 400.

The bag 402 is inserted into the housing 408, such that the housing 408 encloses the bag 402 and the coil assembly 400. The housing 408 protects the bag 402 and the coil assembly 400 from physical damage. Also, the housing 408 includes mounting brackets 410 for mounting the housing 408 to a platform, such as an operating table. In some embodiments, the housing 408 is configured to be situated on a patient. In some embodiments, the housing 408 is one of a box, a patch, a paddle, and a plate, which are configured to be at least one of mounted to a platform and situated on a patient. In some embodiments, the housing 408 is filled with a fluid, such as cooling fluid 112, which surrounds the bag 402 to cool the coil assembly 400.

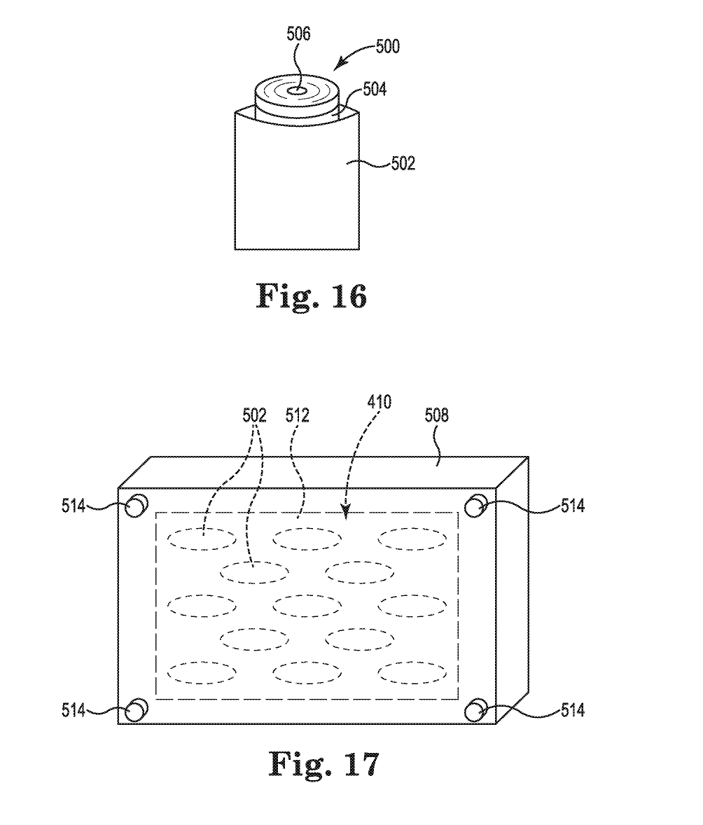

FIG. 16 is a diagram illustrating a coil 500 that is radio-translucent and a bag 502 that is radio-translucent, according to embodiments of the disclosure. The coil 500 is inserted into the bag 502 and the bag 502 is hermetically sealed. Hermetically sealing the bag 502 prevents ingress of moisture into the bag 502, moisture that would otherwise damage the coil 500. In some embodiments, the bag 502 is vacuum sealed. In some embodiments, the bag 502 is filled with an inert gas, such as argon or nitrogen, and then hermetically sealed. In some embodiments, the bag 502 is filled with a fluid, such as cooling fluid 112, to cool the coil 500.

The coil 500 includes conductors 504, such as ribbons or wires, wound around a core 506. Also, in some embodiments, a radio-translucent plug (not shown) is inserted into the core 506 of the coil 500.

The conductors 504 are made out of one or more radio-translucent materials. In some embodiments, the conductors 504 include lithium. In some embodiments, the conductors 504 include a lithium alloy. In some embodiments, the conductors 504 include aluminum. In some embodiments, the conductors 504 include beryllium. In some embodiments, the conductors 504 include carbon nano-tubes. In some embodiments, the coil 500 includes a lithium coil that includes lithium conductors 504, such as ribbons or wires, which are insulated by at least one of a thin film insulator and a silicone insulator. In some embodiments, the coil 500 is similar to one or more of: the coils 102 (shown in FIGS. 2 and 3); the coil 120 of FIG. 4; the coil 140 of FIG. 5A; and the coils 202 (shown in FIGS. 9, 10, 11A, 11B, and 11C).

The bag 502 with the coil 500 inside is radio-translucent. Thus, the bag 502 with the coil 500 inside can be situated in the field of view of a radiograph, such as an x-ray and/or fluoroscopy, without significantly affecting the contrast of the radiographic image of the point of interest, where the radio translucent bag 502 with the radio-translucent coil 500 inside minimizes attenuation in addition to that resulting from the materials of the point of interest, such as a patient and the patient's muscle and/or bone. The bag 502 is one example of a vapor barrier that surrounds or encapsulates the coil 502 and/or a coil assembly to prevent ingress of moisture that would otherwise damage the coil 502 or the coil assembly. In some embodiments, the bag 502 includes plastic. In some embodiments, the bag 502 is an aluminized bag, i.e., a bag at least coated with a layer of aluminum. In some embodiments, the magnetic field generator 22 (shown in FIG. 1) includes multiple radio translucent bags 502, each enclosing a radio-translucent coil 500.

FIG. 17 is a diagram illustrating a housing 508 that is radio-translucent, which encloses a coil assembly 510 that is radio translucent, according to embodiments of the disclosure. The coil assembly 510 includes a coil frame 512 that is radio-translucent and multiple bags 502, each enclosing a coil 500, according to embodiments of the disclosure. The coil assembly 510 can be used for generating and/or sensing magnetic fields. Also, the coil assembly 510 is radio-translucent and can be situated in the field of view of a radiograph, such as an x-ray and/or fluoroscopy, without significantly affecting the contrast of the radiographic image of the point of interest, where the coil assembly 510 minimizes attenuation in addition to that resulting from the materials of the point of interest, such as a patient and the patient's muscle and/or bone. In some embodiments, the coil assembly 510 is similar to one or more of the coil assembly 100 of FIG. 1 and the coil assembly 200 of FIG. 9, with the exception that each coil 500 is inserted into a separate bag 502 and inserted or disposed in the coil frame 512.

The housing 508 with the coil assembly 510, including the coil frame 512 and multiple bags 502 each enclosing a coil 500, is radio-translucent. Thus, the housing 508 and the coil assembly 510 can be situated in the field of view of a radiograph, such as an x-ray and/or fluoroscopy, without significantly affecting the contrast of the radiographic image of the point of interest, where the housing 508 and the coil assembly 510 minimize attenuation in addition to that resulting from the materials of the point of interest, such as a patient and the patient's muscle and/or bone. In some embodiments, the housing 508 includes and/or is made of carbon fiber. In some embodiments, the magnetic field generator 22 (shown in FIG. 1) includes the housing 508 with the coil assembly 510 including the coil frame 512 and multiple bags 502, each enclosing a coil 500.

The housing 508 protects the coil assembly 510, including the coil frame 512 and the multiple bags 502 and coils 500, from physical damage. Also, the housing 508 includes mounting brackets 514 for mounting the housing 508 to a platform, such as an operating table. In some embodiments, the housing 508 is configured to be situated on a patient. In some embodiments, the housing 508 is one of a box, a patch, a paddle, and a plate, which are configured to be at least one of mounted to a platform and situated on a patient. In some embodiments, the housing 508 is filled with a fluid, such as cooling fluid 112, which surrounds the coil assembly 510 to cool the coil assembly 510.

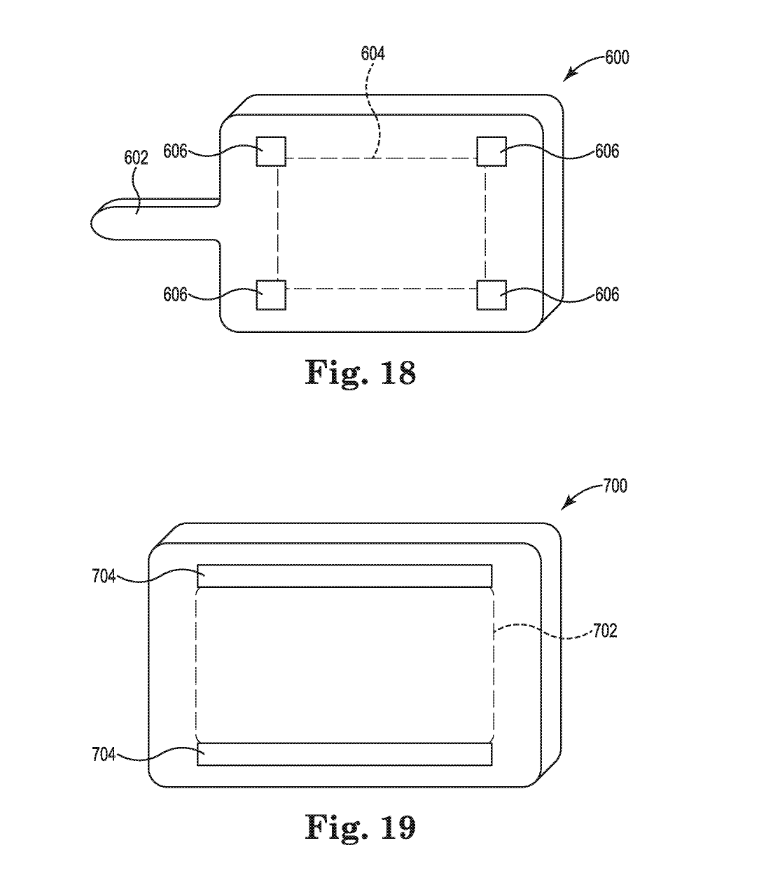

FIG. 18 is a diagram illustrating a paddle 600 that is radio-translucent, according to embodiments of the disclosure. The paddle 600 includes a handle 602 for moving the paddle 600 from one location to another and an active area 604 for generating and/or sensing magnetic fields. The paddle 600 encloses and protects a radio-translucent coil assembly including coils in a coil frame, as described above in relation to the housings 408 and 508. Also, the coil assembly can be inserted into a radio-translucent bag or each of the coils can be individually and separately inserted into radio-translucent bags, as described above. In some embodiments, the paddle 600 is similar to the housing 408 (shown in FIG. 15), except it is shaped like a paddle. In some embodiments, the paddle 600 is similar to the housing 508 (shown in FIG. 17), except it is shaped like a paddle.

The paddle 600 can be placed under the patient, on the patient, or attached to a platform, such as an operating table. The paddle 600 includes mounting devices 606. In some embodiments, the mounting devices 606 are adhesive mounts for attaching the paddle 600 to the patient and/or the platform. In some embodiments, the mounting devices 606 are mounting brackets for attaching the paddle 600 to a platform.

The paddle 600 can be situated in the field of view of a radiograph, such as an x-ray and/or fluoroscopy, without significantly affecting the contrast of the radiographic image of the point of interest, where the radio-translucent paddle 600 minimizes attenuation in addition to that resulting from the materials of the point of interest, such as a patient and the patient's muscle and/or bone. In some embodiments, the paddle 600 includes and/or is made of carbon fiber. In some embodiments, the magnetic field generator 22 (shown in FIG. 1) includes the paddle 600.

FIG. 19 is a diagram illustrating a patch 700 that is radio-translucent, according to embodiments of the disclosure. The patch 700 includes an active area 702 for generating and/or sensing magnetic fields. The patch 700 encloses and protects a radio-translucent coil assembly including coils in a coil frame, as described above in relation to housings 408 and 508. Also, the coil assembly can be inserted into a radio-translucent bag or each of the coils can be individually and separately inserted into radio-translucent bags, as described above. In some embodiments, the patch 700 is similar to the housing 408 (shown in FIG. 15), except it is flexible. In some embodiments, the patch 700 is similar to the housing 508 (shown in FIG. 17), except it is flexible.

The patch 700 can be placed under the patient, on the patient, or attached to a platform, such as an operating table. The patch 700 includes mounting devices 704. In some embodiments, the mounting devices 704 are adhesive patches for attaching the patch 700 to the patient and/or the platform. In some embodiments, the mounting devices 704 are mounting brackets for attaching the patch 700 to a platform.

The patch 700 can be situated in the field of view of a radiograph, such as an x-ray and/or fluoroscopy, without significantly affecting the contrast of the radiographic image of the point of interest, where the radio-translucent patch 700 minimizes attenuation in addition to that resulting from the materials of the point of interest, such as a patient and the patient's muscle and/or bone. In some embodiments, the patch 700 includes and/or is made of carbon fiber. In some embodiments, the magnetic field generator 22 (shown in FIG. 1) includes the patch 700.

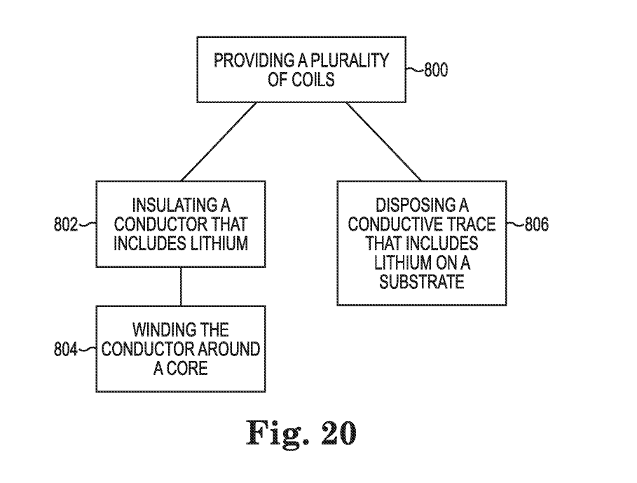

FIG. 20 is a diagram illustrating a method of manufacturing a system that includes coils that are radio-translucent, according to embodiments of the disclosure. At 800, the method includes the step of providing a plurality of coils that are radio-translucent.

At 802 and 804, providing the plurality of coils includes the steps of insulating at least one conductor that includes lithium (802), and winding the conductor, such as in circles, around a core (804) to provide one of the plurality of coils. In some embodiments, insulating the at least one conductor includes at least one of insulating the at least one conductor with a thin film insulator and insulating the at least one conductor with a silicone insulator. In some embodiments, the method includes disposing the coil into a coil frame that is radio-translucent such that the coil and at least a portion of the coil frame have a uniform radio-translucency in at least one direction. In some embodiments, the method includes inserting a plug that is radio-translucent into the core of the coil to provide uniform radio-translucency in at least one direction. In some embodiments the method includes at least one of: inserting the coil frame with the coil into a bag that is radio-translucent, hermetically sealing the bag to prevent moisture ingress, and inserting the bag into a housing that is radio translucent; and inserting the coil into a separate bag that is radio-translucent, hermetically sealing the separate bag to prevent moisture ingress, and inserting the separate bag into the housing that is radio-translucent.

At 806, providing the plurality of coils includes the step of disposing a conductive trace that includes lithium on a substrate to provide a planar coil that is radio-translucent. In some embodiments the method includes the step of disposing the conductive trace on the substrate such that the planar coil and at least a portion of the substrate have a uniform radio-translucency in at least one direction. In some embodiments, the method includes the step of inserting the substrate into a bag that is radio-translucent, hermetically sealing the bag to prevent moisture ingress, and inserting the bag into a housing that is radio translucent.

Various modifications and additions can be made to the exemplary embodiments discussed without departing from the scope of the disclosure. For example, while the embodiments described above refer to particular features, the scope of this disclosure also includes embodiments having different combinations of features and embodiments that do not include all of the described features. Accordingly, the scope of the disclosure is intended to embrace all such alternatives, modifications, and variations as fall within the scope of the claims, together with all equivalents thereof.

We claim:

1. A system comprising:
at least one coil that is radio-translucent and includes at least one conductor that includes lithium.

2. The system of claim 1, wherein the at least one conductor is a lithium conductor that is wrapped around a core of the at least one coil.

3. The system of any of claims 1 and 2, wherein the at least one conductor is insulated by at least one of a thin film insulator and a silicone insulator.

4. The system of any of claims 1-3, wherein the at least one coil includes a coating to prevent moisture ingress.

5. The system of claim 1, wherein the at least one coil is a planar coil.

6. The system of any of claims 1-5, comprising at least one of a coil frame and a substrate that supports the at least one coil.

7. The system of claim 6, wherein the at least one of the coil frame and the substrate is radio-translucent and at least a portion of the at least one of the coil frame and the substrate and the at least one coil have a uniform radio-translucency in at least one direction.

8. The system of any of claims 6 and 7, comprising at least one vapor barrier that is radio-translucent and one of vacuum sealed and filled with an inert gas and sealed, wherein one of:
the at least one of the coil frame and the substrate and the at least one coil are situated inside the at least one vapor barrier and the at least one vapor barrier is hermetically sealed to prevent ingress of moisture; and
each coil of the at least one coil is situated in a separate vapor barrier of the at least one vapor barrier and the separate vapor barrier is hermetically sealed to prevent ingress of moisture.

9. The system of claim 8, comprising a housing that is radio-translucent, wherein the at least one vapor barrier is situated in the housing.

10. The system of any of claims 1-9, wherein different coils of the at least one coil are oriented in at least two different directions.

11. A method of manufacturing a system, the method comprising at least one of:
insulating at least one conductor that includes lithium and winding the at least one conductor around a core to provide a coil that is radio-translucent; and
disposing a conductive trace that includes lithium on a substrate to provide a planar coil that is radio-translucent.

12. The method of claim 11, comprising at least one of:
disposing the coil into a coil frame that is radio-translucent such that the coil and at least a portion of the coil frame have a uniform radio-translucency in at least one direction; and
disposing the conductive trace on the substrate such that the planar coil and at least a portion of the substrate have a uniform radio-translucency in at least one direction.

13. The method of claim 12, comprising inserting a plug that is radio-translucent into a core of the coil to provide the uniform radio-translucency in the at least one direction.

14. The method of any of claims 12 and 13, comprising at least one of:
inserting at least one of the coil frame and the substrate into a vapor barrier that is radio-translucent, hermetically sealing the vapor barrier to prevent moisture ingress, and inserting the vapor barrier into a housing that is radio translucent; and
inserting the coil into a separate vapor barrier that is radio-translucent, hermetically sealing the separate vapor barrier to prevent moisture ingress, and inserting the separate vapor barrier into the housing that is radio-translucent.

15. The method of any of claims 11-14, wherein insulating the at least one conductor comprises at least one of insulating the at least one conductor with a thin film insulator and insulating the at least one conductor with a silicone insulator.

16. A system comprising:
a plurality of coils, wherein the plurality of coils are radio-translucent to x-rays and include conductors that include lithium.

17. The system of claim 16, comprising a coil frame that supports the plurality of coils, wherein the coil frame and the plurality of coils provide a uniform radio-translucent image to x-rays in at least one direction.

18. The system of claim 16, wherein each of the plurality of coils includes a conductor of the conductors that is electrically insulated by at least one of a thin film insulator and a silicone insulator.

19. The system of claim 16, wherein at least one of the plurality of coils is a planar coil that includes a conductive trace that includes lithium.

20. The system of claim 16, comprising a housing that is radio-translucent, wherein the plurality of coils are situated inside the housing.

21. The system of claim 20, wherein the housing is at least one of a box, a patch, a paddle, and a plate that is configured to be at least one of mounted to a platform and situated on a patient.

22. The system of claim 16, comprising at least one vapor barrier that is radio-translucent, wherein one of:
the plurality of coils are situated inside the at least one vapor barrier and the at least one vapor barrier is hermetically sealed to prevent ingress of moisture; and
each of the plurality of coils is individually situated inside a separate vapor barrier of the at least one vapor barrier and the separate vapor barrier is hermetically sealed to prevent ingress of moisture.

23. The system of claim 22, wherein the at least one vapor barrier is at least one of vacuum sealed, filled with an inert gas and sealed, and an aluminized bag.

24. The system of claim 22, comprising a housing that is radio-translucent, wherein the at least one vapor barrier is situated inside the housing.

25. A system comprising:
a coil assembly, including:
a coil frame; and
a plurality of coils that are radio-translucent and supported by the coil frame;
a vapor barrier that is radio-translucent, wherein at least one of the plurality of coils is situated in the vapor barrier; and
a housing that is radio-translucent, wherein the vapor barrier is situated in the housing.

26. The system of claim 25, wherein the coil frame and the plurality of coils provide a uniform radio-translucent image in at least one direction.

27. The system of claim 25, wherein the plurality of coils are configured for at least one of generating a magnetic field and sensing a magnetic field and the plurality of coils are configured to be at least one of individually activated and activated as part of a group of the plurality of coils.

28. The system of claim 25, wherein all of the plurality of coils are oriented in the same direction.

29. The system of claim 25, wherein different coils of the plurality of coils are oriented in at least two different directions.

30. A method of manufacturing a system, the method comprising:
providing a plurality of coils by at least one of:
insulating a conductor that includes lithium and winding the conductor around a core to provide a coil that is radio-translucent; and
disposing a conductive trace that includes lithium on a substrate to provide a planar coil that is radio-translucent.

31. The method of claim 30, comprising:
disposing the plurality of coils in a coil frame to provide a coil assembly that provides a uniform radio-translucency in at least one direction.

32. The method of claim 31, comprising:
inserting a plug that is radio-translucent in a core of one or more of the plurality of coils to provide the uniform radio-translucency.

33. The method of claim 30, wherein insulating a conductor comprises at least one of insulating the conductor with a thin film insulator and insulating the conductor with a silicone insulator.

34. The method of claim 30, comprising at least one of:
inserting the plurality of coils into a vapor barrier that is radio-translucent and hermetically sealing the vapor barrier to prevent moisture ingress; and
inserting each of the plurality of coils into a separate vapor barrier that is radio-translucent and hermetically sealing each of the separate vapor barriers to prevent moisture ingress.

35. The method of claim 34, comprising at least one of:
inserting the vapor barrier with the plurality of coils into a housing that is radio-translucent; and
inserting at least one of the separate vapor barriers into the housing that is radio-translucent.
